(19) United States
(12) Patent Application Publication
(10) Pub. No.: US 2021/0085432 A1
(43) Pub. Date: Mar. 25, 2021

(54) Cosmetic Facial Prosthesis and Methods for Making the Same

(71) Applicant: Kimberly C. Sheets, Akron, OH (US)

(72) Inventor: Kimberly C. Sheets, Akron, OH (US)

(21) Appl. No.: 17/028,572

(22) Filed: Sep. 22, 2020

Related U.S. Application Data
(60) Provisional application No. 62/903,991, filed on Sep. 23, 2019.

(51) Int. Cl. A61C 13/01; A61C 13/225

(52) U.S. Cl. CPC A61C 13/01; A61C 13/2255

(57) ABSTRACT

The present disclosure relates to a cosmetic facial or maxillary prosthesis. The cosmetic facial or maxillary prosthesis is designed to be worn by individuals having certain physical deformities that affect the appearance of their face as a temporary cosmetic piece that provides an aesthetically improved appearance by softening and straightening certain facial imperfections. The cosmetic facial or maxillary prosthesis is fully functional in that it stays in place with normal movement of the user's mouth, for example, while talking, eating and chewing and does not interfere with daily tasks. The cosmetic facial maxillary prosthesis is not outwardly observable by others and may be removed by the user at any time.

### I. BACKGROUND

### A. Technical Field

The present disclosure relates to a cosmetic device for improving the appearance of a person's facial features.

### B. Description of Related Art

People with facial deformities often face difficult social situations in public either in the form of unwanted attention, general poor treatment or rejection by others, shunning, ridicule, self-consciousness or perceived inadequacies.

These facial deformities may be the result of aging, stroke, genetic diseases or certain conditions such as bell's palsy. For example, bell's palsy is a neurological disorder which results in the muscles on one side of a person's face becoming weak or paralyzed. Because bell's palsy affects only one side of the face, the affected side may droop or become stiff while the non-affected side is perfectly normal. Another condition which results in drooping of the face is patients who have suffered a stroke. Other facial deformities such as crooked mouth cause a person's lips or mouth to slant towards one side. Also, aging in general causes the epidermis of the skin to become thinner and also causes the collagen and elastic tissue within the skin to degrade and hang loosely. As a result, the skin around the face may begin to sag, stretch and become drawn in with age.

Options to correct such deformities within the medical field can be expensive, personally invasive and not practical for people having such conditions. Consequently, more often than not, people with such conditions do not desire to undergo extensive medical treatment or surgeries to correct any such existent or perceived facial deformities. Rather, what people affected with such conditions often desire is a product that can provide a temporary cosmetic fix to their deformity, whether existent or perceived.

Accordingly, what is needed in the art is a device that functions as a cosmetic facial or maxillary prosthesis to correct such deformities by filling in drawn in, drooping, stiff, or slanted facial features and softening facial imperfections. The device would be placed within the inside of a person's mouth or oral cavity and can be worn temporarily while the person goes out in public to attend events, parties and any other types of social gatherings or to carry out simply daily tasks. The device would be easily removable by the user whenever he or she wishes to take the device out from the oral cavity and would function solely as a cosmetic piece, that is, although it may, the device need not provide any medical, health related or dental benefit. These and other advantages are provided in the cosmetic facial or maxillary prosthesis provided in the present disclosure.

### II. SUMMARY

Provided is a cosmetic facial prosthesis. The cosmetic facial prosthesis includes a periodontal prosthetic having a front side and a back side that fits over a user's upper or lower jaw bone, wherein the front side of the periodontal prosthetic fits over the user's front gingivae and the back side of the periodontal prosthetic fits over the back side of the user's back gingivae; at least one opening within the periodontal prosthetic which fits over one or more of a user's teeth as the periodontal prosthetic lies over a user's gingivae; a wire or wire-like support structure embedded within the periodontal prosthetic that provides structural support to the cosmetic facial prosthesis; a hook which extends from the periodontal prosthetic to attach the cosmetic facial prosthesis to a user's teeth or to the upper or lower jaw bone of the user, wherein the hook is in the form of a resin structure, a resin structure mimicking the appearance of the user's gingivae, a wire or wire-like structure embedded within a resin structure so as to not be visible or positioned on the back side of the user's mouth so as to not be visible.

Provided is a cosmetic facial prosthesis. The cosmetic facial prosthesis includes a periodontal prosthetic having a front side and a back side that fits over a user's upper or lower jaw bone, wherein the front side of the periodontal prosthetic fits over the user's front gingivae and the back side of the periodontal prosthetic fits over the back side of the user's back gingivae; at least one opening within the periodontal prosthetic which is capable of fitting over one or more of a user's teeth as the periodontal prosthetic lies over a user's gingivae; and a convex portion on the periodontal prosthetic that extends in a distal direction toward the gingiva and extends outward within the vestibule in a buccal direction.

According to certain aspects of the present teaching, the periodontal prosthetic includes a left side, a center side and a right side, wherein the left side includes a front left side and a back left side, the center side includes an optional front center side and a back center side and the right side includes a front right side and a back right side.

According to certain aspects of the present teaching, the cosmetic facial prosthesis includes an internal hook which extends within the periodontal prosthetic or an external hook which extends outward from the periodontal prosthetic and which is capable of attaching the cosmetic facial prosthesis to a user's tooth or to the upper or lower jaw bone of the user.

According to certain aspects of the present teaching, the cosmetic facial prosthesis further includes a wire or wire-like support structure embedded within the periodontal prosthetic that provides structural support to the cosmetic facial prosthesis.

According to certain aspects of the present teaching, the internal or external hook is in the form of a resin structure, a resin structure mimicking the appearance of the user's gingivae, a wire or wire-like structure embedded within a resin structure so as to not be visible or positioned on the back side of the user's mouth so as to not be visible or is a metal structure which extends outward from the periodontal prosthetic.

According to certain aspects of the present teaching, the front center side is excluded and the back center side includes a wire or wire-like structure that connects the left side of the cosmetic facial prosthesis to the right side of the cosmetic facial prosthesis.

According to certain aspects of the present teaching, the cosmetic facial prosthesis includes a first opening between the front and back left side of the cosmetic facial prosthesis and a second opening between the front and back right side of the cosmetic facial prosthesis, wherein the first opening extends the entire length of the left side of the cosmetic facial prosthesis and the second opening extends the entire length of the right side of the cosmetic facial prosthesis.

According to certain aspects of the present teaching, the cosmetic facial prosthesis further includes a plurality of individual tooth openings between the front and back left side of the cosmetic facial prosthesis and a plurality of individual tooth openings between the front and back right side of the cosmetic facial prosthesis that allow the cosmetic facial prosthesis to fit over individual teeth.

According to certain aspects of the present teaching, the periodontal prosthetic includes a left side, a right side, a front side and a back side that is capable of fitting over the upper or lower jaw bone within the right side, center side or left side of a buccal cavity.

According to certain aspects of the present teaching, the cosmetic facial prosthesis further includes a first external hook which extends outward from the back right side of periodontal prosthetic and a second external hook which extends outward from the back left side of the periodontal prosthetic, wherein the first and second external hooks are capable of attaching the cosmetic facial prosthesis to a user's tooth or to the upper or lower jaw bone of the user.

According to certain aspects of the present teaching, the cosmetic facial prosthesis further includes an opening extending the entire length of the cosmetic facial prosthesis or a plurality of individual tooth openings between the front and back side of the cosmetic facial prosthesis that allow the cosmetic facial prosthesis to fit over individual teeth and wherein the cosmetic facial prosthesis optionally comprises an internal hook which extends within the opening spanning the entire length of the cosmetic facial prosthesis or which extends between the individual tooth openings between the front and back side of the cosmetic facial prosthesis, optionally, wherein the hook is connected to a wire or wire-like extension which extends from the back left or back right side of the cosmetic facial prosthesis, wherein the internal or external hook and wire or wire-like extension is in the form of a resin structure, a resin structure mimicking the appearance of the user's gingivae, a wire or wire-like structure embedded within a resin structure so as to not be visible or positioned on the back side of the user's mouth so as to not be visible or is a metal structure which extends outward from the periodontal prosthetic.

According to certain aspects of the present teaching, the cosmetic facial prosthesis, the front center side or a portion of the front center side, front left side or front right side is excluded and the back center side includes a connecting surface or a wire or wire-like structure that connects the left side of the cosmetic facial prosthesis to the right side of the cosmetic facial prosthesis, further including an opening which i) extends the entire length of the left side and/or right side of the cosmetic facial prosthesis or ii) a plurality of individual tooth openings between the front and back left side of the cosmetic facial prosthesis and/or a plurality of individual tooth openings between the front and back right side of the cosmetic facial prosthesis that allow the cosmetic facial prosthesis to fit over individual teeth, further including a first external hook which extends outward from the front right side of periodontal prosthetic and a second external hook which extends outward from the front left side of the periodontal prosthetic, wherein the first and second external hooks are capable of attaching the cosmetic facial prosthesis to a user's tooth or to the upper or lower jaw bone of the user, wherein the external hooks are in the form of a resin structure, a resin structure mimicking the appearance of the user's gingivae, a wire or wire-like structure embedded within a resin structure so as to not be visible or is a metal structure which extends outward from the periodontal prosthetic.

According to certain aspects of the present teaching, the cosmetic facial prosthesis further includes a top surface teeth covering which extends over the left side, the center side and right side of the periodontal prosthetic and connects to the front and back left side, front and back center side and front and back right side of the periodontal prosthetic, further including a front surface teeth covering and a back surface teeth covering which extends downward from the top surface teeth covering, wherein the cosmetic facial prosthesis includes a bottom opening which renders the cosmetic facial prosthesis capable of fitting over the teeth and upper or lower jaw bone, optionally, wherein the cosmetic facial prosthesis further includes an opening within a portion of the front side capable of exposing one or more teeth.

According to certain aspects of the present teaching, the front side opening is within the front center side, wherein a center connector portion lies below the front center side opening and connects the left side of the prosthetic facial prosthesis to the right side of the prosthetic facial prosthesis.

According to certain aspects of the present teaching, the cosmetic facial prosthesis further includes a convex portion positioned below the front surface teeth covering or upward above the front surface teeth covering depending on whether the prothesis is placed on the lower or upper jaw, wherein the convex portion extends in a distal direction toward the gingiva and extends outward within the vestibule in a buccal direction.

According to certain aspects of the present teaching, the cosmetic facial prosthesis, the convex portion extends outward from the bottom surface of the teeth covering at at least one angle, wherein the at least one angle is at least about 100° or more.

According to certain aspects of the present teaching, the cosmetic facial prosthesis, the front left side, front center side and front right side and convex portion of the periodontal prosthesis comprises a single thickness or a plurality of different thicknesses, wherein the thickness includes about 3 mm or more.

According to certain aspects of the present teaching, the front center side is excluded and the back side comprises a concave connecting surface that connects the left side of the cosmetic facial prosthesis to the right side of the cosmetic facial prosthesis, wherein the convex portion extends along the entire length of the left side and the entire length of the right side of the periodontal prosthetic, wherein the convex portion extends outward from the bottom surface of the teeth covering at least one angle, wherein the at least one angle is at least about 100° or more, and wherein the front left side, front center side and front right side and convex portion of the periodontal prosthesis comprises a single thickness or a plurality of different thicknesses, wherein the thickness comprises about 3 mm or more.

According to certain aspects of the present teaching, the front center side is excluded and the back side comprises a wire or wire-like connector that connects the left side of the cosmetic facial prosthesis to the right side of the cosmetic facial prosthesis, wherein the convex portion extends along the entire length of the left side and the entire length of the right side of the periodontal prosthetic, wherein the periodontal prosthesis includes at least one internal hook which extends within the space between the front left side and the back left side and/or the space between the front right side and back right side of the periodontal prosthesis, wherein the wire or wire-like connector and/or the internal hooks are in the form of a resin structure, a resin structure mimicking the appearance of the user's gingivae, a wire or wire-like structure embedded within a resin structure so as to not be visible or is a metal structure, and wherein the cosmetic facial prosthesis does not include a top surface teeth covering, a front surface teeth covering and a back surface teeth covering.

According to certain aspects of the present teaching, the convex portion extends along the entire length of the left side and the entire length of the right side of the periodontal prosthetic, wherein the convex portion extends outward from the bottom surface of the teeth covering at least one angle, wherein the at least one angle is at least about 100° or more, and wherein the front left side, front center side and front right side and convex portion of the periodontal prosthesis comprises a single thickness or a plurality of different thicknesses, wherein the thickness comprises about 3 mm or more.

### III. BRIEF DESCRIPTION OF THE DRAWINGS

FIG. 1 is a front perspective view of a human mouth.

FIG. 2 is a first illustrative embodiment of a cosmetic facial or maxillary prosthesis.

FIG. 3 is a second illustrative embodiment of a cosmetic facial or maxillary prosthesis.

FIG. 4 is a third illustrative embodiment of a cosmetic facial or maxillary prosthesis.

FIG. 5 is a fourth illustrative embodiment of a cosmetic facial or maxillary prosthesis.

FIG. 6 is a fifth illustrative embodiment of a cosmetic facial or maxillary prosthesis.

FIG. 7 is a sixth illustrative embodiment of a cosmetic facial or maxillary prosthesis.

FIG. 8 is a seventh illustrative embodiment of a cosmetic facial or maxillary prosthesis.

FIG. 9 is an eighth illustrative embodiment of a cosmetic facial or maxillary prosthesis.

FIG. 10 is a ninth illustrative embodiment of a cosmetic facial or maxillary prosthesis.

FIG. 11a shows a tenth illustrative embodiment of a cosmetic facial or maxillary prosthesis.

FIG. 11b shows an eleventh illustrative embodiment of a cosmetic facial or maxillary prosthesis.

FIGS. 12a and 13a show a twelfth illustrative embodiment of a cosmetic facial or maxillary prosthesis.

FIGS. 12b and 13b show a thirteenth illustrative embodiment of a cosmetic facial or maxillary prosthesis.

FIGS. 14a and 15a show a fourteenth illustrative embodiment of a cosmetic facial or maxillary prosthesis.

FIGS. 14b and 15b show a fifteenth illustrative embodiment of a cosmetic facial or maxillary prosthesis.

FIGS. 16A-16G show a sixteenth illustrative embodiment of a cosmetic facial or maxillary prosthesis.

FIGS. 17A-17G show a seventeenth illustrative embodiment of a cosmetic facial or maxillary prosthesis.

FIGS. 18A-18G show an eighteenth illustrative embodiment of a cosmetic facial or maxillary prosthesis.

FIGS. 19A-19G show a nineteenth illustrative embodiment of a cosmetic facial or maxillary prosthesis.

### III. DETAILED DESCRIPTION

Provided is a cosmetic facial or maxillary prosthesis. The cosmetic facial or maxillary prosthesis may be worn by individuals suffering from certain facial deformities including but not limited to bell's palsy, crooked mouth as well as by people who have suffered the effects of stroke, aging and any other condition, disorder or genetic disease. The cosmetic facial or maxillary prosthesis may be worn temporarily by such individuals as they go out in public to complete daily tasks and attend social gatherings such as events, parties, dinners, etc. The cosmetic facial or maxillary prosthesis is intended as a temporary correction softening certain deformities within the face and allowing the person wearing it to look or appear in an aesthetically pleasing way to one's self and others while presenting himself or herself to others or to the public in general. When no longer desired or needed, for example, when the social event is over or the daily task requiring public appearance is completed, the person wearing the device may easily remove the cosmetic facial or maxillary prosthesis without hassle. The cosmetic facial or maxillary prosthesis is designed to be stable and fully functional within the person's mouth. That is, it will not fall out of place while the person is wearing the device and will not hinder the person wearing the device from undertaking normal activities such as talking and eating. The person wearing the device does not have to worry about the device falling out of his or her mouth or out of place while talking, making certain facial expressions or while eating. Moreover, the device is designed to be unnoticeable while within the person's mouth. That is the person may smile and talk while showing his or her teeth without the device being noticeable. Also, the attaching parts of the device including the wire or wire-like structure are either embedded within a gum like structure so as to be hidden or are positioned behind the user's teeth so as to not be visible.

Referring now to the drawings wherein the illustrations shown therein are for the purpose of illustrating embodiments of the invention only and are not for the purpose of limiting the same, and wherein like reference numerals are understood to refer to like components.

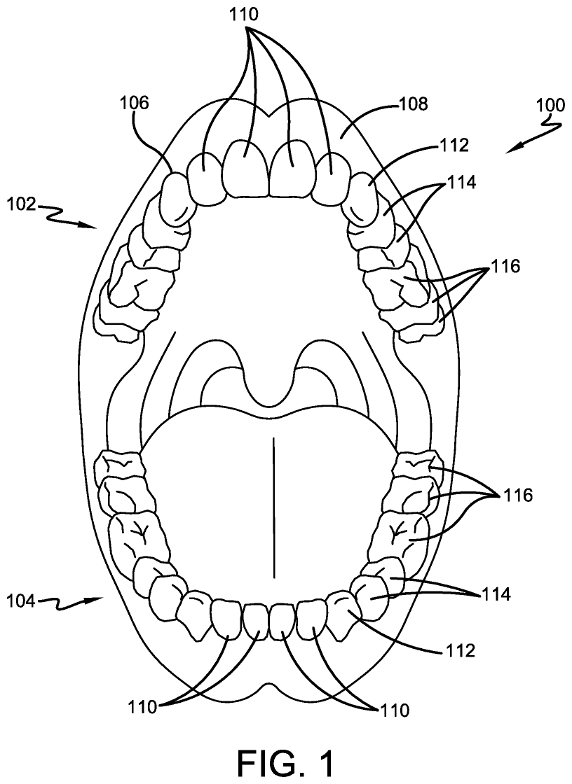

FIG. 1 illustrates the oral cavity of a typical human mouth which may house the cosmetic facial prosthesis, also referred to as a cosmetic maxillary prosthesis. As shown within FIG. 1, the mouth 100 includes an upper jaw 102 and a lower jaw 104. The teeth 106 extend from the dental alveolus or gums 108 of the upper and lower jaw 102 and 104. The teeth 106 for each jaw 102 and 104 include four incisors 110, two canines (also referred to as cuspids) 112, four premolars (also referred to as bicuspids) 114, and six molars 116 on each of the upper and lower jaws.

The cosmetic facial or maxillary prothesis may be designed to fit on one side or both sides of the inside a person's mouth or oral cavity. As such, it may be symmetrical or non-symmetrical in shape and design. It may also be designed to fit over the teeth and on the upper jaw bone or lower jaw bone of a person to account for certain imperfections within the oral cavity. The cosmetic facial prothesis generally includes a periodontal prosthetic or gum-like structure. The periodontal prosthetic may be made out of a resin or plastic material that mimics the color of a person's gums or gingivae. The periodontal prosthetic is designed and may be customized to correct imperfections within the oral cavity by pushing certain areas of the buccal mucosa outward through areas or portions having various shapes and thicknesses to shape an individual's face and provide an aesthetically appealing outward appearance. In certain cases, when positioned on the lower jaw, the periodontal prosthetic can push down on the lower lip to soften certain facial imperfections.

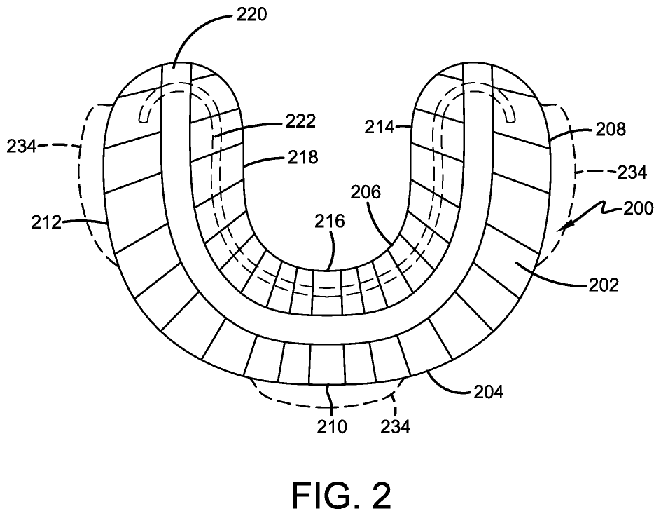

FIG. 2 provides a first embodiment of the cosmetic facial or maxillary prosthesis 200.

The cosmetic facial prosthesis of FIG. 2 is symmetrical in shape and is designed to fit on the upper or lower jaw bone to correct any visible facial deformities on both sides of the face. As shown within FIG. 2, the cosmetic facial prosthesis includes a periodontal prosthetic 202 that is symmetrical in shape. The periodontal prosthetic 202 generally includes a front side 204 and a back side 206 that fits over the upper or lower jaw bone. It also includes a front left side 208 that fits over the left side of the upper or lower jaw bone, a front center side 210 that fits over the front portion of the upper or lower jaw bone, a front right side 212 that fits over the right side of the upper or lower jaw bone, a back left side 214 that fits over the left side of the upper or lower jaw bone, a back center side 216 that fits over the center front portion upper or lower jaw bone and a back right side 218 that fits over the right side of the upper or lower jaw bone. The front portion of the periodontal prosthetic 202 may also include a convex portion 234. The convex portion may be positioned at any point along the front surface of the periodontal prosthetic 202 and may have any length. A non-limiting example of a convex portion 234 positioned at three different areas (i.e., the front left side 208, the front center side 210 and the front right side 208) of the front surface of the periodontal prosthetic is illustrated within FIG. 2. The cosmetic facial prosthesis 200 as shown in FIG. 2 also includes opening or cavity 220 that extends from the left side to the right side of the cosmetic facial prosthesis and which is positioned between the front side and the back side of the periodontal prosthetic 202. This opening or cavity 220 is designed to fit over a person's teeth as the periodontal prosthetic 202 lies over a person's gums or gingivae. The back side of the periodontal prosthetic may include a wire or wire-like structural support 222 to provide structural support to the cosmetic facial prosthesis. In certain embodiments, the wire or wire-like structural support 222 may be made from metal, plastic or any other sufficiently rigid structure capable of providing structural support to the cosmetic facial prosthesis 200. The wire or wire-like structural support is embedded within the periodontal prosthetic 202 and is not visible. Also, the wire or wire-like structural support 222 may be designed to hook around the back end of the cosmetic facial prosthesis around the back tooth and/or over the jaw bone to provide a means for attaching and/or securing the cosmetic facial prosthesis 202 to the tooth or jaw bone of the user.

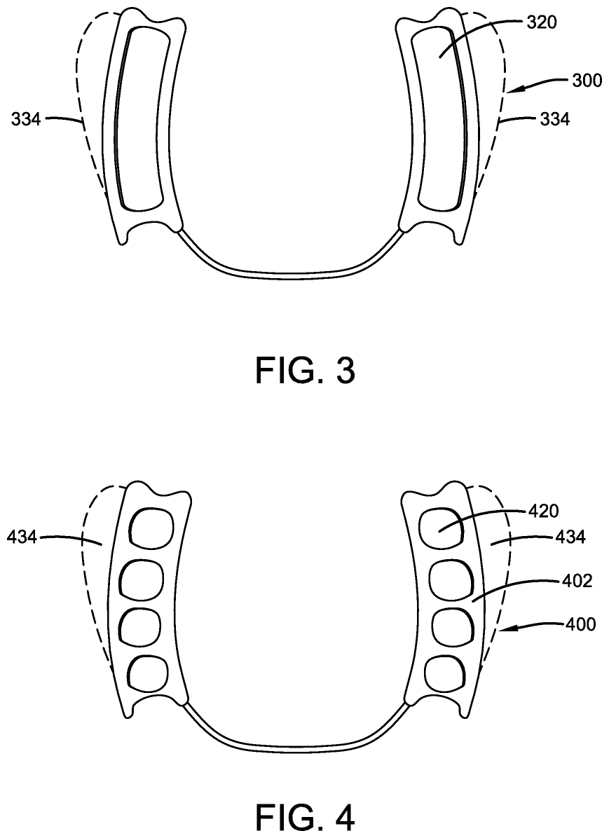

FIG. 3 provides an alternative embodiment of the cosmetic facial or maxillary prosthesis 300. The cosmetic facial prosthesis of FIG. 3 is symmetrical in shape and is designed to fit on the upper or lower jaw bone to correct any visible facial deformities on both sides of the face. As shown within FIG. 3, the cosmetic facial prosthesis includes a periodontal prosthetic 302 that is symmetrical in shape. The periodontal prosthetic 302 generally includes a front side and a back side that fits over the upper or lower jaw bone. The cosmetic facial prosthesis generally includes a front left side that fits over the upper or lower left jaw bone, a front right side that fits over the upper or lower right jaw bone, a back left side that fits over the upper or lower jaw bone, a back center side that fits over the upper or lower jaw bone and a back right side that fits over the upper or lower jaw bone. The front portion of the periodontal prosthetic 302 may also include a convex portion 334. The convex portion may be positioned at any point along the front surface of the periodontal prosthetic 302 and may have any length. A non-limiting example of a convex portion 334 positioned at two different areas (i.e., the front left side and the front right side) of the front surface of the periodontal prosthetic is illustrated within FIG. 3. The cosmetic facial prosthesis 300 as shown in FIG. 3 also includes opening or cavity 320 that extends on the left side and the right side of the cosmetic facial prosthesis and which is positioned between the front side and the back side of the periodontal prosthetic 302. This opening or cavity 320 is designed to fit over a person's teeth as the periodontal prosthetic 302 lies over a person's gums or gingivae. The back side of the periodontal prosthetic may include a wire or wire-like structural support to provide structural support to the cosmetic facial prosthesis. In certain embodiments, the wire may be made from metal, plastic or any other sufficiently rigid structure capable of providing structural support to the cosmetic facial prosthesis 300. As with the embodiment of FIG. 1, the wire may be embedded within the periodontal prosthetic 302 and is not visible. However, the wire may also serve as a connecting piece linking the front right side to the front left side of the cosmetic facial prosthesis 300 as shown in FIG. 3. Also, the wire may be designed to hook around the back end of the cosmetic facial prosthesis around the back tooth and/or over the jaw bone (not shown) to provide a means for attaching and/or securing the cosmetic facial prosthesis 302 to the tooth or jaw bone of the user.

FIG. 4 provides an alternative embodiment of the cosmetic facial or maxillary prosthesis 400 which is similar in design to FIG. 3. The front portion of the periodontal prosthetic 402 may also include a convex portion 434. The convex portion may be positioned at any point along the front surface of the periodontal prosthetic 402 and may have any length. A non-limiting example of a convex portion 434 positioned at two different areas (i.e., the front left side and the front right side) of the front surface of the periodontal prosthetic is illustrated within FIG. 4. One difference between the embodiment shown in FIG. 4 from that of FIGS. 2 and 3 is that instead of utilizing an opening or cavity 220 or 320 which is designed to fit over a person's teeth as the periodontal prosthetic 302 lies over a person's gums or gingivae, the periodontal prosthetic 402 includes individual or separate tooth openings 420 that allow the cosmetic facial prosthesis to fit over the user's teeth in a glove-like manner. In addition, it is also contemplated that the concept of individual or separate tooth openings 420 within the periodontal prosthetic may also be incorporated within FIG. 2.

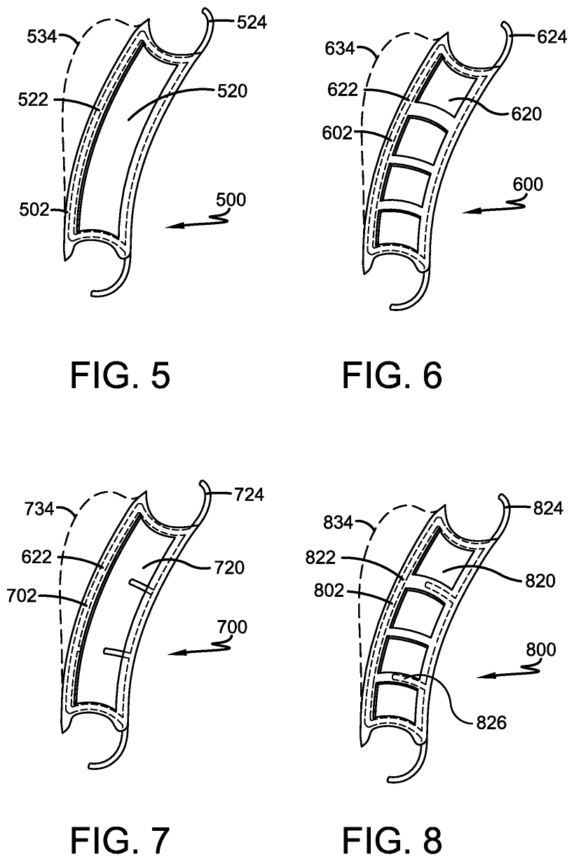

FIG. 5 provides an alternative embodiment of the cosmetic facial or maxillary prosthesis 500 having an opening or cavity 520 which is designed to fit over a person's teeth as the periodontic prosthetic 502 lies over a person's gums or gingivae. The cosmetic facial prosthesis 500 is non-symmetrical in shape and design so as to fit on one side or within a certain portion or position within a person's mouth. For example, the cosmetic facial prosthesis 500 may fit within the left side, front side, or right side of a person's upper or lower jaw. The front portion of the periodontal prosthetic 502 may also include a convex portion 534. The convex portion may be positioned at any point along the front surface of the periodontal prosthetic 502 and may have any length. A non-limiting example of a convex portion 534 positioned at the front surface of the periodontal prosthetic is illustrated within FIG. 5. The cosmetic facial prosthesis 500 also includes a wire or wire-like structural support 522 embedded within the periodontal prosthetic 502 so as not to be visible. In certain embodiments, the wire or wire-like structural support may be embedded throughout the periodontic prosthetic 502 including the front portion, side portions and back portion as shown in FIG. 5 although in other embodiments, the wire or wire-like structural support may only be present within a certain portion of the of the periodontic prosthetic, for example, the back portion of the periodontic prosthetic. The wire or wire-like structural support may extend out from the back side of the periodontic prosthetic as shown within FIG. 5 in the form of a hook or hook-like structure to attach the cosmetic facial prosthesis to the back side of a person's teeth or to the upper jaw or lower jaw bone of the person wearing the device to secure the device to the user. In certain embodiments, the wire or wire-like structural support that extends out from the periodontic prosthetic in a hook or hook-like structure 524 may have a color and texture which mimics that of a person's gingiva or gingivae. It is also contemplated that the concept of the hook or hook like structure within the periodontal prosthetic may also be incorporated within FIGS. 2, 3, 4 or any of the other embodiments disclosed herein.

FIG. 6 illustrates an alternative embodiment of the cosmetic facial or maxillary prosthesis similar to that shown in FIG. 5 in that it includes a periodontal prosthetic 602, a wire or wire-like structural support 622 within the periodontal prosthetic 602, a hook or hook like structure 624 and is designed to fit over one side or within a certain portion or position within a person's mouth, with the exception that instead of utilizing an opening or cavity 520 which is designed to fit over a person's teeth as the periodontal prosthetic lies over a person's gums or gingivae, the periodontal prosthetic 602 includes individual or separate tooth openings 620 that allow the cosmetic facial prosthesis to fit over the user's teeth in a glove-like manner. In addition, the front portion of the periodontal prosthetic 602 may also include a convex portion 634. The convex portion may be positioned at any point along the front surface of the periodontal prosthetic 602 and may have any length. A non-limiting example of a convex portion 634 positioned on the front surface of the periodontal prosthetic is illustrated within FIG. 6.

FIG. 7 illustrates an alternative embodiment of the cosmetic facial or maxillary prosthesis similar to that shown in FIG. 5 in that it includes a periodontal prosthetic 702, a wire or wire-like structural support 722 within the periodontal prosthetic 702, a hook or hook-like structure 724 and is designed to fit over one side or within a certain portion or position within a person's mouth with the exception that the wire or wire-like structure does not extend out from opposing ends of the periodontal prosthetic 702 within the hook or hook-like structure 724 to hook around the user's teeth or upper or lower jaw bone. Instead, the wire or wire-like structure 722 extends around and the opening 720. In addition, the prosthesis includes two internal hook or hook-like structures 726 which fit between the user's teeth or over the upper or lower jaw bone within the opening 720. Depending on the particular embodiment utilized, the wire or wire-like structures 722 may or may not extend into the internal hook or hook-like structures. In other embodiments, the internal hook or hook-like structures 726 may be composed of the wire or wire-like structure 722. The front portion of the periodontal prosthetic 702 may also include a convex portion 734. The convex portion may be positioned at any point along the front surface of the periodontal prosthetic 702 and may have any length. A non-limiting example of a convex portion 734 positioned on the front surface of the periodontal prosthetic is illustrated within FIG. 7.

FIG. 8 illustrates an alternative embodiment of the cosmetic facial or maxillary prosthesis similar to that shown in FIG. 7 in that it includes a periodontal prosthetic 802, a wire or wire-like structural support 822 within the periodontal prosthetic 802, a hook or hook-like structure 824 and is designed to fit over one side or within a certain portion or position within a person's mouth with the exception that instead of utilizing an opening or cavity 720 which is designed to fit over a person's teeth as the periodontal prosthetic lies over a person's gums or gingivae, the periodontal prosthetic 802 includes individual or separate tooth openings 820 that allow the cosmetic facial prosthesis 800 to fit over the user's teeth in a glove-like manner. In addition, the prosthesis includes two internal hook or hook-like structures 826 which fit between the user's teeth or over the upper or lower jaw bone within the opening 820. Depending on the articular embodiment utilized, the wire or wire-like structures 822 may or may not extend into the internal hook or hook-like structures. In other embodiments, the internal hook or hook-like structures 726 may be composed of the wire or wire-like structure 822 as shown in FIG. 8. The front portion of the periodontal prosthetic 802 may also include a convex portion 834. The convex portion may be positioned at any point along the front surface of the periodontal prosthetic 802 and may have any length. A non-limiting example of a convex portion 834 positioned on the front surface of the periodontal prosthetic is illustrated within FIG. 8.

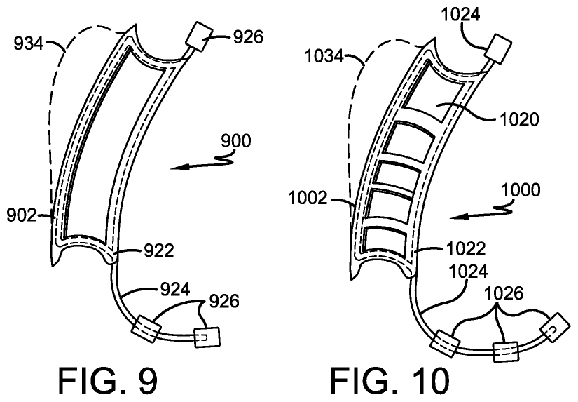

FIG. 9 illustrates an alternative embodiment of the cosmetic facial or maxillary prosthesis 900 similar to that shown in FIG. 5 except that the wire or wire-like structure 922 forms an extension 924 which extends out from one of the opposing ends of the periodontal prosthetic of the cosmetic facial prosthesis 900. The extension 924 is intended to be designed as long as required to reach a gap between a user's teeth where a hook or hook-like structure 926 may attach between the user's teeth or over the upper or lower jaw bone of the user. In addition, the front portion of the periodontal prosthetic 902 may also include a convex portion 934. The convex portion may be positioned at any point along the front surface of the periodontal prosthetic 902 and may have any length. A non-limiting example of a convex portion 934 positioned on the front surface of the periodontal prosthetic is illustrated within FIG. 9.

FIG. 10 illustrates an alternative embodiment of the cosmetic facial or maxillary prosthesis 1000 similar to that shown in FIG. 9 in that the wire or wire-like structure 1022 also forms an extension 1024 which extends out from one of the opposing ends of the periodontal prosthetic of the cosmetic facial prosthesis 1000. The extension 1024 is intended to be designed as long as required to reach a gap between a user's teeth where a hook or hook-like structure 1026 may attach between the user's teeth or over the upper or lower jaw bone of the user. FIG. 10 differs from FIG. 9 in that it includes individual or separate tooth openings 1020 that allow the cosmetic facial prosthesis 1000 to fit over the user's teeth in a glove-like manner. In addition, the front portion of the periodontal prosthetic 1002 may also include a convex portion 1034. The convex portion may be positioned at any point along the front surface of the periodontal prosthetic 1002 and may have any length. A non-limiting example of a convex portion 1034 positioned on the front surface of the periodontal prosthetic is illustrated within FIG. 10.

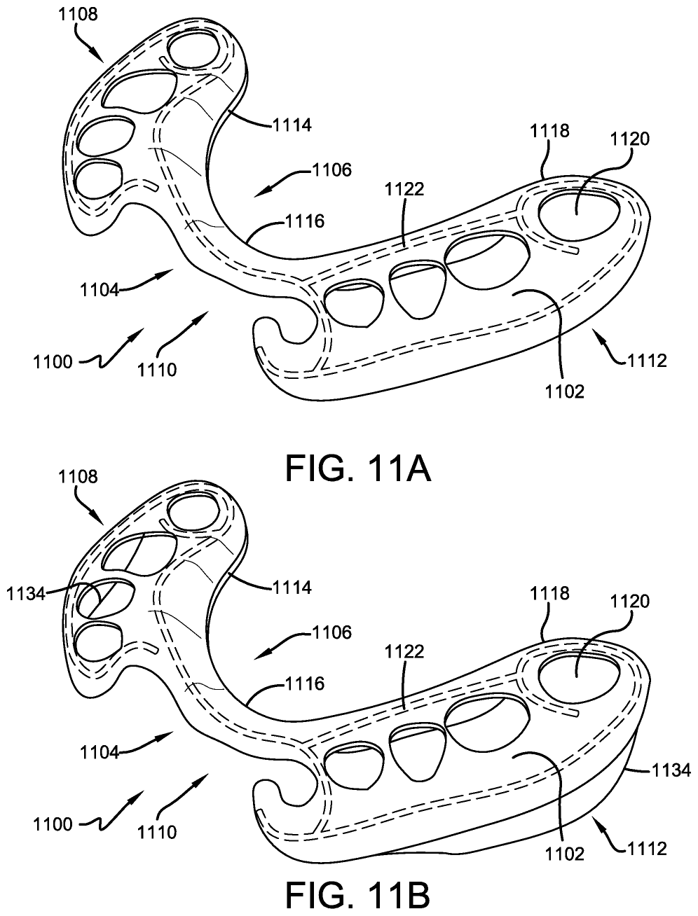

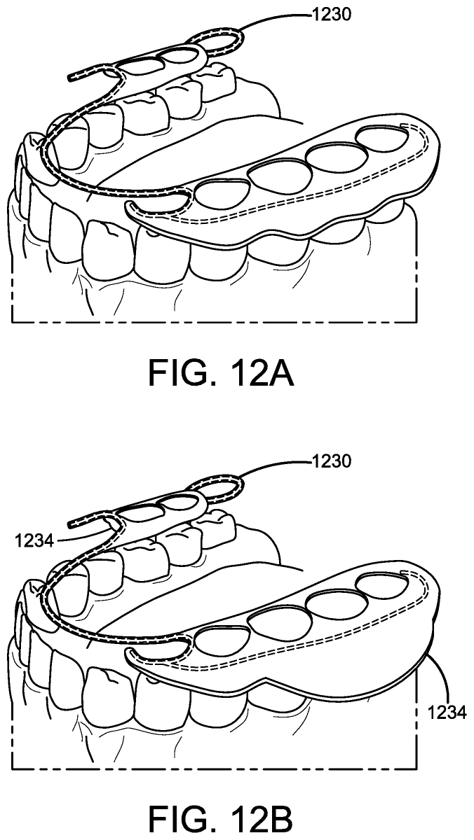

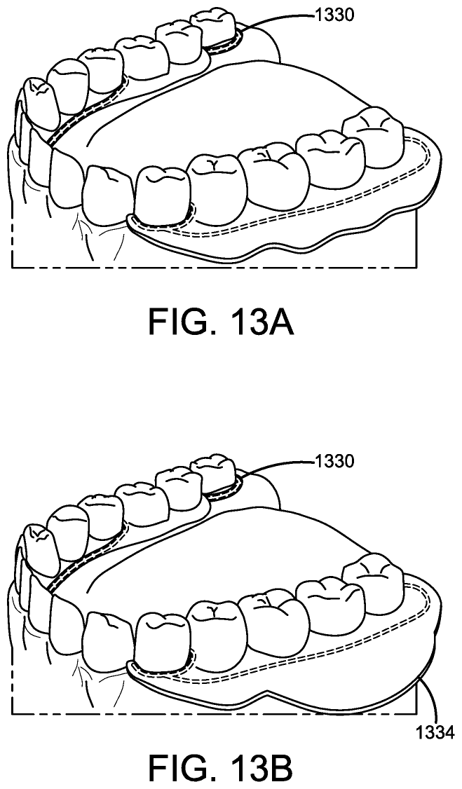

FIG. 11a illustrates an alternative embodiment of the cosmetic facial or maxillary prosthesis 1100. The cosmetic facial prosthesis of FIG. 11a is symmetrical in shape and is designed to fit on the upper or lower jaw bone to correct any visible facial deformities on both sides of the face. As shown within FIG. 11a, the cosmetic facial prosthesis includes a periodontal prosthetic 1102 that is symmetrical in shape. As in FIG. 2, the periodontal prosthetic 1102 of FIG. 11a generally includes a front side 1104 and a back side 1106 that fits over the upper or lower jaw bone. It also includes a front left side 1108 that fits over the upper or lower left jaw bone, a front center side 1110 that fits over the upper or lower jaw bone, a front right side 1112 that fits over the upper or lower right jaw bone, a back left side 1114 that fits over the upper or lower jaw bone, a back center side 1116 that fits over the upper or lower jaw bone and a back right side 1118 that fits over the upper or lower jaw bone. The front center side 1110 of the periodontal prosthetic 1102 includes cutout section in cases where a portion of the periodontal prosthetic 1102 is not required to correct a deformity at the front of the user's mouth or jaw line. The cosmetic facial prosthesis 1100 also includes separate tooth openings or cavities 1120 that are designed to fit over a person's teeth in a glove-like manner as the periodontal prosthetic 1102 lies over a person's gums or gingivae. The back side of the periodontal prosthetic may include a wire or wire-like structural support 1122 to provide structural support to the cosmetic facial prosthesis. In certain embodiments, the wire 1122 may be made from metal, plastic or any other sufficiently rigid structure capable of providing structural support to the cosmetic facial prosthesis 1100. The wire or wire-like structural support is embedded within the periodontal prosthetic 1102 and is not visible. Also, the wire or wire-like structural support 1122 may be designed to hook around the back end of the cosmetic facial prosthesis around the back tooth and/or over the jaw bone to provide a means for attaching and/or securing the cosmetic facial prosthesis 1102 to the tooth or jaw bone of the user. In an alternative embodiment, FIG. 12a illustrates the cosmetic facial prosthesis positioned over but not attached to the teeth whereas FIG. 13a illustrates the cosmetic facial prosthesis attached to the teeth. In certain embodiments, a hook 1230/1330 may be positioned outside of the periodontal prosthetic to attach to the user's tooth and secure the cosmetic facial prosthesis in place. FIGS. 11b, 12b, and 13b illustrate a non-limiting example of a variation of the prosthesis shown in FIGS. 11a, 12a, and 13a wherein the front portion of the periodontal prosthetic includes a convex portion 1134, 1234 and 1334. The convex portion may be positioned at any point along the front surface of the periodontal prosthetic (e.g., the front left or front right side of the periodontal prosthetic) and may have any length.

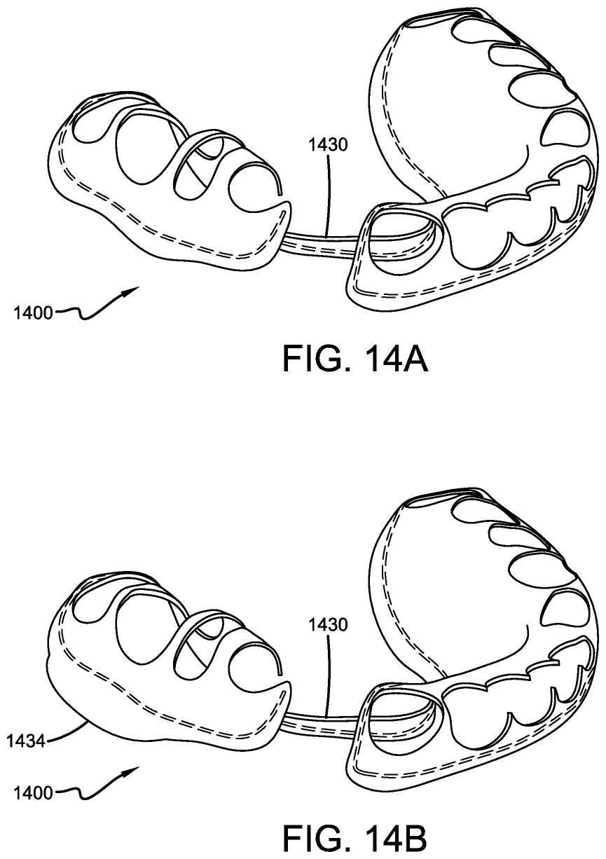

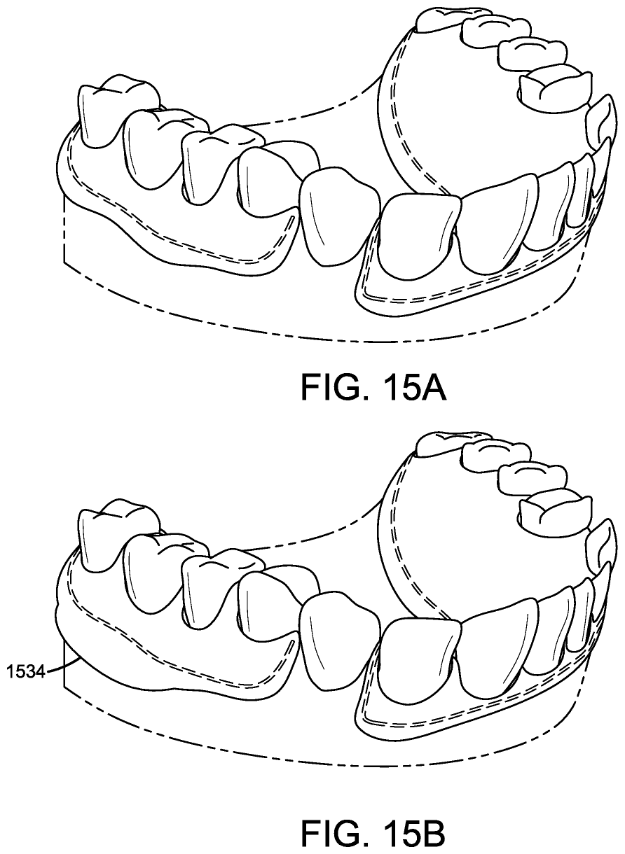

FIGS. 14a and 15a illustrate an alternative embodiment of the cosmetic facial or maxillary prosthesis 1400. The cosmetic facial prosthesis of FIGS. 14a and 15a is non-symmetrical in shape to account for non-symmetrical deformities within a user's face and is designed to fit on the upper or lower jaw bone. The cosmetic facial prosthesis is also designed to correct any visible facial deformities on one or both sides of the face. As shown within FIGS. 14a and 15a, the cosmetic facial prosthesis includes similar components to other exemplary embodiments except that the cosmetic facial prosthesis includes a back side structural support 1430 connecting gaps within the periodontal prosthetic where cosmetic facial correction is not needed. FIG. 15a illustrates the cosmetic facial prosthesis of FIG. 14a applied over a user's teeth. FIGS. 14b and 15b illustrate a non-limiting example of an alternative embodiment wherein the front portion of the periodontal prosthetic includes a convex portion 1434 and 1534. The convex portion may be positioned at any point along the front surface of the periodontal prosthetic (e.g., the front left, front center or front right side portion of the periodontal prosthetic) and may have any length.

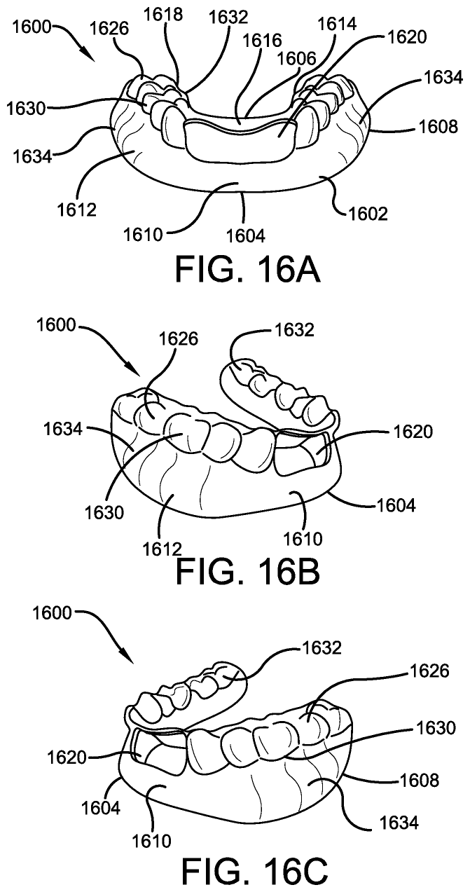

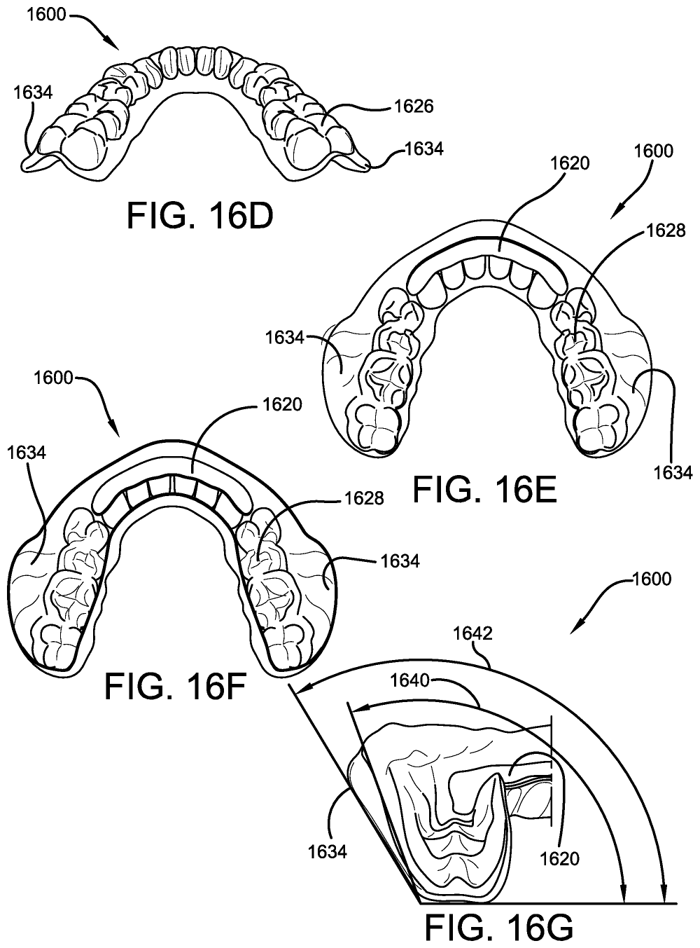

FIGS. 16A through 16G illustrate an alternative embodiment of the cosmetic facial or maxillary prosthesis 1600. The cosmetic facial prosthesis of FIGS. 16A through 16F is symmetrical in shape and is designed to fit over the user's teeth on the upper or lower jaw bone to correct any visible facial deformities on both sides of the face. As shown within FIGS. 16A through 16F, the cosmetic facial prosthesis includes a periodontal prosthetic 1602 that is symmetrical in shape. The periodontal prosthetic 1602 generally includes a front side 1604 and a back side 1606 that fits over the upper or lower jaw bone. It also includes a front left side 1608 that fits over the left side of the upper or lower jaw bone, a front center side 1610 that fits over the front portion of the upper or lower jaw bone, a front right side 1612 that fits over the right side of upper or lower jaw bone, a back left side 1614 that fits over the left side of the upper or lower jaw bone, a back center side 1616 that fits over the center portion of the upper or lower jaw bone, a back right side 1618 that fits over the right side upper or lower jaw bone and a teeth covering portion having a top surface 1626, a bottom surface 1628, a front side surface 1630 and a back side surface 1632. The cosmetic facial prosthesis 1600 as shown in FIGS. 16A through 16F includes an opening or cavity 1620 within the front center side 1610. Below the opening is a center connector portion 1624 which connects the left side of the prosthetic facial prosthesis to the right side of the prosthetic facial prosthesis. This opening or cavity 1620 is designed to fit over a person's teeth as the periodontal prosthetic 1602 lies over a person's gums or gingivae and allows for exposure of the front teeth which most commonly will be the user's incisors 110 although other teeth may also be exposed depending on the size of the user's teeth and the size of the opening depending on the particular embodiment employed. The periodontal prosthetic 1602 includes a convex portion 1634 positioned below or above the portion of the periodontal prosthetic 1602 that covers the teeth depending on whether the periodontal prosthetic fits over the lower or upper jaw. The convex portion 1634 extends downward below the teeth or upward above the teeth depending on whether the prothesis is placed on the lower or upper jaw in a distal direction toward the gingiva and extends outward within the vestibule (the area between the inside of the cheek and the gums) in the buccal direction towards the inner cheek when worn. In certain embodiments, the convex portion extends outward from the bottom surface of the teeth covering at an angle 1640 of at least about 100° or more while in other embodiments, the convex portion may extend outward from the bottom surface of the teeth covering at an angle 1640 of at least about 110° or more, an angle of at least about 120° or more, or an angle of at least about 125° or more. An example of the various angles of the convex portion with respect to the bottom side 1628 of the teeth covering portion is illustrated in the non-limiting embodiment illustrated within FIG. 16G.

It is noted that the angles of the convex portion with respect to other features of the cosmetic facial prosthesis may vary as the device may be customized to a person's specific needs. For example, the angle of the convex portion with respect to the bottom surface of the teeth covering may vary to be greater than 90° of the teeth covering portion of the device at various points within the buccal cavity, for example, in the buccal direction adjacent to the gingiva below any one or more of the molars, premolars, bicuspids, cuspids and incisors on one, both or any side of the jaw when worn. As shown within FIG. 16G, the convex portion 1634 includes two different angles (a first angle 1640 at about 100° and a second angle 1642 at about) 120° which extend outward within the vestibule in the buccal direction towards the inner cheek.

However, convex portion 1634 which extends from the bottom surface of the teeth covering may include any number of angles as needed (e.g., one, two, three or more angles) to fill in the deformity within the buccal region of a particular person.

As further shown within FIGS. 16E and 16F which illustrates the bottom of the cosmetic or facial prosthesis 1600, the front left side 1608, the front center side 1610 and the front right side 1612 has a thickness that extends in the buccal direction adjacent to the gingiva when worn. This thickness may be consistent throughout the front left side 1608, front center side 1610 and front right side 1612 of the prosthesis or it may vary depending on the shape of the person's gingiva, teeth and jaw bone and degree of deformity that is intended to be corrected with the convex portion 1634 of the device. It is therefore, contemplated that the thickness the front left side 1608, front center side 1610, front right side 1612 and convex portion 1634 of the prosthesis may vary below any one or more of the molars, premolars, bicuspids, cuspids and incisors on one, both or any side of the jaw and that the prothesis may have one or a plurality of different thicknesses including from one to sixteen or more. In certain embodiments, the thickness within these regions of the device may be about 1 mm or more, however, it is contemplated that the thickness is generally about 3 mm or more for purpose correcting a particular facial deformity. With respect to the convex portion 1634, the thickness may range from about 3 mm to about 5 mm or more or from about 3 mm to about 7 mm or more. In further embodiments, the thickness of the convex portion 1634 may range from about 3 mm to about 10 mm or about 15 mm or more.

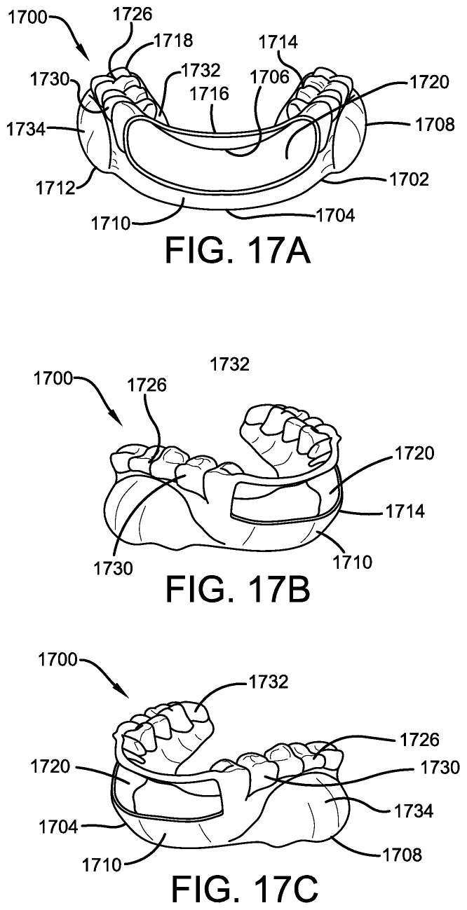

FIGS. 17A through 17F illustrate an alternative embodiment of the cosmetic facial or maxillary prosthesis 1700. The cosmetic facial prosthesis of FIGS. 17A through 17F is symmetrical in shape and is designed to fit over the user's teeth on the upper or lower jaw bone to correct any visible facial deformities on both sides of the face. As shown within FIGS. 17A through 17F, the cosmetic facial prosthesis includes a periodontal prosthetic 1702 that is symmetrical in shape. The periodontal prosthetic 1702 generally includes a front side 1704 and a back side 1706 that fits over the upper or lower jaw bone. It also includes a front left side 1708 that fits over the left side of the upper or lower jaw bone, a front center side 1710 that fits over the front portion of the upper or lower jaw bone, a front right side 1712 that fits over the right side of upper or lower jaw bone, a back left side 1714 that fits over the left side of the upper or lower jaw bone, a back center side 1716 that fits over the center portion of the upper or lower jaw bone, a back right side 1718 that fits over the right side upper or lower jaw bone, and a teeth covering portion having a top surface 1726, a bottom surface 1728, a front side surface 1730 and a back side surface 1732. The cosmetic facial prosthesis 1700 as shown in FIGS. 17A through 17F includes an opening or cavity 1720 within the front center side 1710. Below the opening is a center connector portion 1724 which connects the left side of the prosthetic facial prosthesis to the right side of the prosthetic facial prosthesis. This opening or cavity 1720 is designed to fit over a person's teeth as the periodontal prosthetic 1702 lies over a person's gums or gingivae and allows for exposure of the front teeth which most commonly will be the user's incisors 110 and cuspids 112 and possibly bicuspids 114 although other teeth may also be exposed depending on the size of the user's teeth and the size of the opening depending on the particular embodiment employed. The periodontal prosthetic 1702 includes a convex portion 1734 positioned below or above the portion of the periodontal prosthetic 1702 that covers the teeth. The convex portion 1734 is positioned below or above the teeth covering portion of the prosthetic (depending on whether the device is intended for the upper or lower jaw) and has an amorphous rectangular-like shape that extends from the teeth covering in a distal direction toward the gingiva and extends outward within the vestibule in the buccal direction towards the inner cheek when worn. In certain embodiments, the convex portion 1734 extends outward from the bottom surface of the teeth covering at an angle of at least about 100° or more while in other embodiments, the convex portion 1734 may extend outward from the bottom surface of the teeth covering at an angle 1740 of at least about 110° or more, an angle of at least about 120° or more, or an angle of at least about 125° or more. An example of the various angles of the convex portion 1734 with respect to the bottom side 1728 of the teeth covering portion is illustrated in the non-limiting embodiment illustrated within FIG. 17G which illustrates a first angle 1740 at about 103° and a second angle 1742 at about 124°.

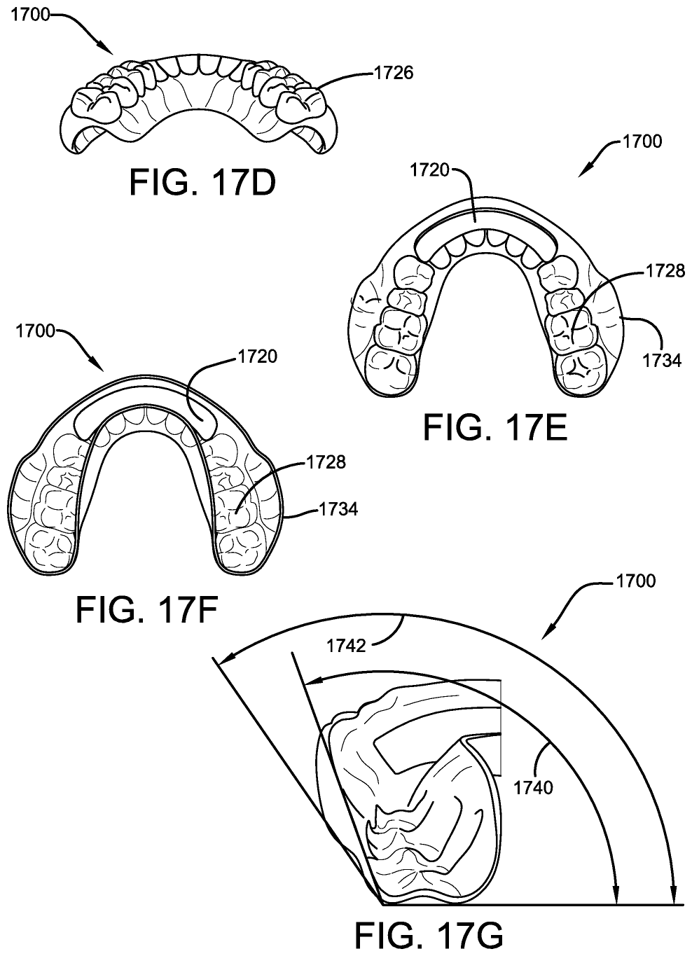

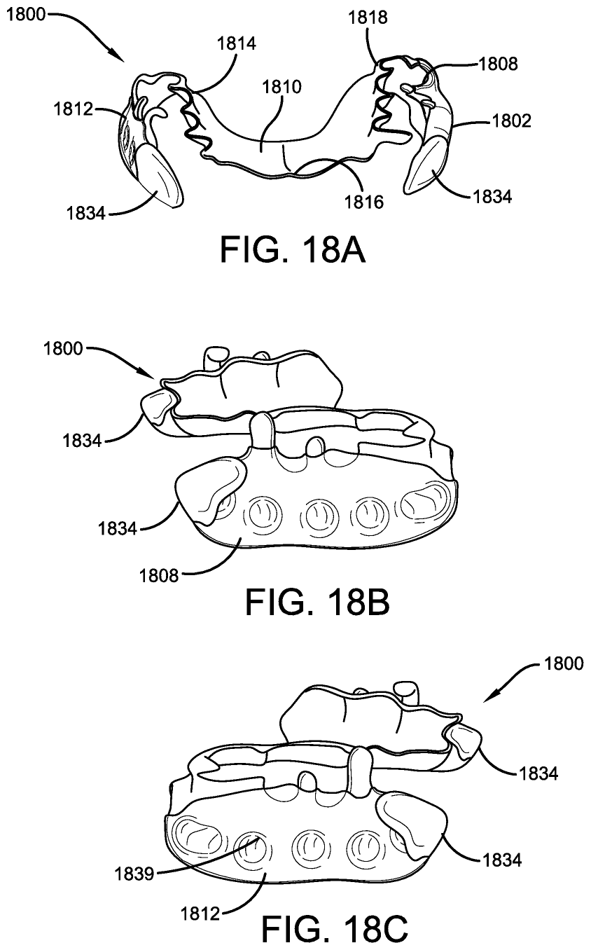

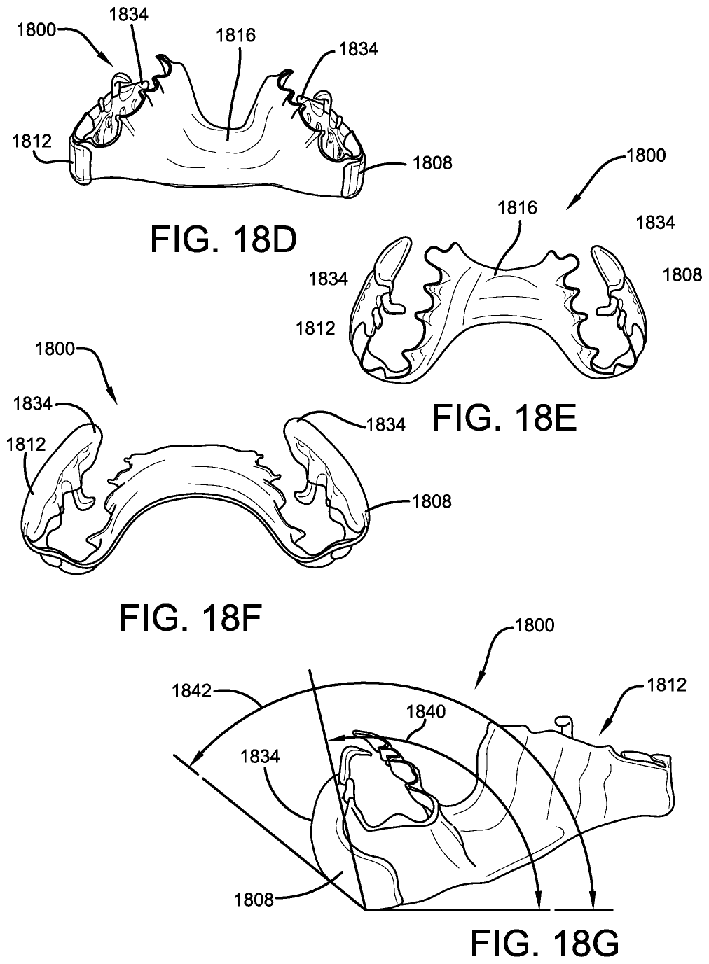

FIGS. 18A through 18F illustrate an alternative embodiment of the cosmetic facial or maxillary prosthesis 1800. The cosmetic facial prosthesis of FIGS. 18A through 18F is symmetrical in shape and is designed to fit on the upper or lower jaw bone to correct any visible facial deformities on both sides of the face. As shown within these figures, the cosmetic facial prosthesis includes a periodontal prosthetic 1802 that is symmetrical in shape. The periodontal prosthetic 1802 generally includes a front side 1804 and a back side 1806 that fits over the upper or lower jaw bone. It also includes a front left side 1808 that fits over the upper or lower left jaw bone, a center surface 1810 that fits over the upper or lower palate, a front right side 1812 that fits over the upper or lower right jaw bone, a back left side 1814 that fits over the upper or lower jaw bone, a back concave center side 1816 that fits over the upper or lower palate and a back right side 1818 that fits over the upper or lower jaw bone. The front side 1804 of the periodontal prosthetic 1802 includes cutout section in cases where a portion of the periodontal prosthetic 1802 is not required to correct a deformity at the front of the user's mouth or jaw line. The cosmetic facial prosthesis 1800 also includes separate tooth openings or cavities 1820 which include an outline on the front side 1804 and back side 1806 in the shape of individual teeth that are designed to fit over a person's teeth in a glove-like manner as the periodontal prosthetic 1802 lies over a person's gums or gingivae. It is noted that the outline may include cut-in portions 1822 or vertical portions 1824 which are designed to fit between individual teeth. The periodontal prosthetic 1802 also includes at least one hook 1826 which may be designed to hook around or over a particular tooth. As shown in FIGS. 18A through 18F, such hooks may be located at the back end of the cosmetic facial prosthesis or along the sides of the cosmetic facial prosthesis to hook and secure the device over any tooth. The periodontal prosthetic 1802 also includes a convex portion 1834 which lies within a substantially horizontal plane. The convex portion 1834 may include all of the features described above with respect to FIGS. 16A through 16G and FIGS. 17A through 17G. In the embodiment shown in FIGS. 18A through 18G, the convex portion extends from the front side 1804 to the back side 1806 along the left side 1808 and the right side 1812 of the periodontal prosthetic 1802, has a thickness ranging from about 1 mm to about 5 mm or more from the front side 1804 to the back side 1806 along the left side 1808 and the right side 1812 of the periodontal prosthetic and has at least one angle 1840 with respect the bottom of the periodontal prosthetic ranging from about 100° or more to about 135° or more. In particular, the embodiment shown in FIG. 18G shows that the periodontal prosthetic 1802 has a first angle 1840 of about 103° and a second angle 1842 of about 135° from the bottom 1830 of the periodontal prosthetic.

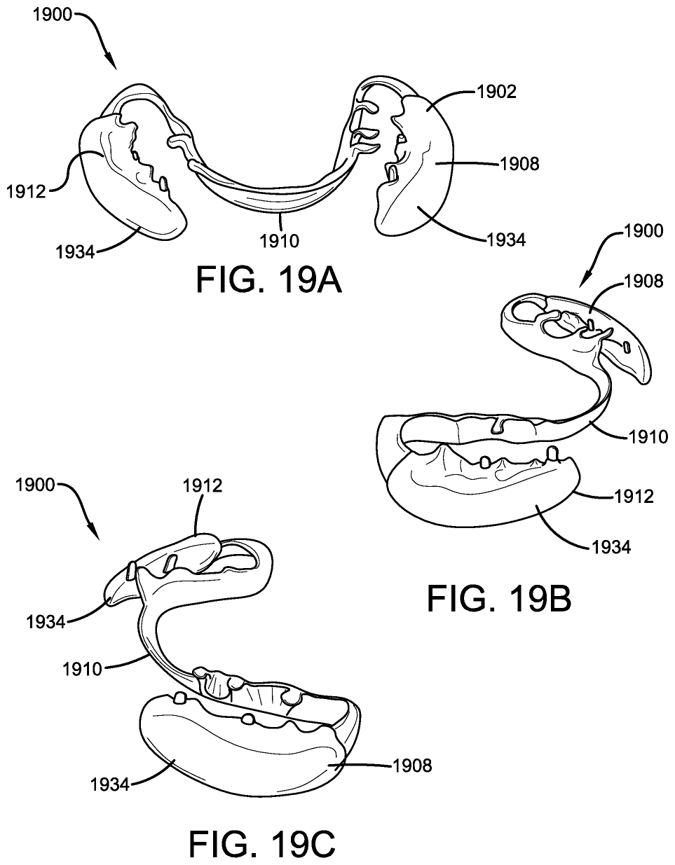

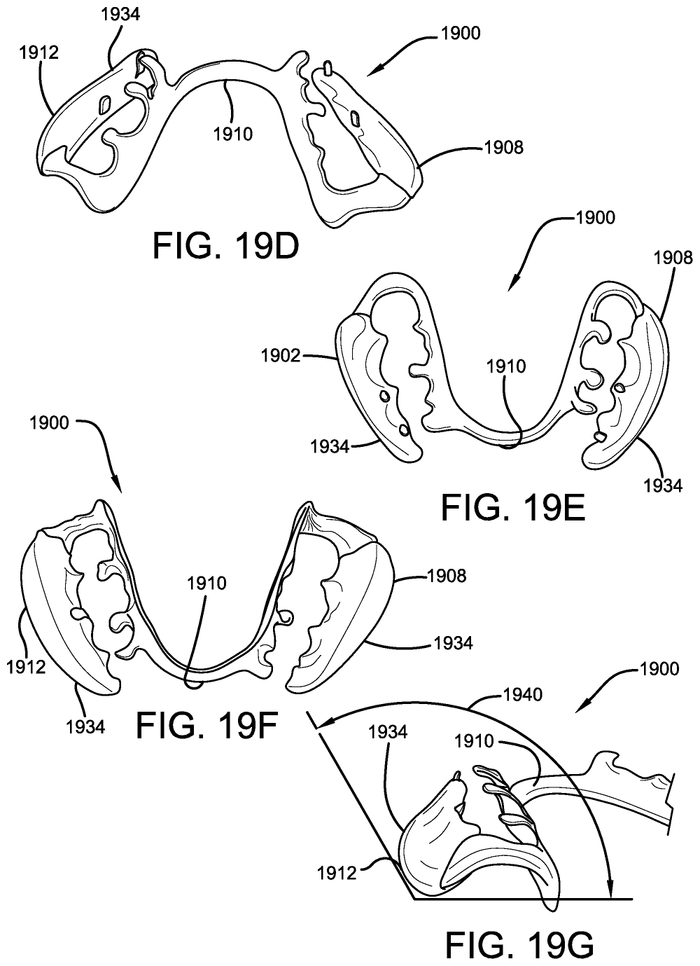

The embodiment illustrated within FIGS. 19A through 19G of the cosmetic facial or maxillary prosthesis 1900 illustrate similar features to that of FIGS. 18A through 18G with the exception that the center surface 1810 is replaced with a center portion connector 1910 along the back center portion 1916 connecting the left side 1908 to the right side 1912 of the periodontal prosthetic 1902. FIG. 19G illustrates that the device having at least one angle 1940 with respect the bottom of the periodontal prosthetic of about 120° or more. In certain embodiments, the center portion connector may be in the form of a resin structure, a resin structure mimicking the appearance of the user's gingivae, a wire or wire-like structure embedded within a resin structure so as to not be visible or positioned on the back side of the user's mouth so as to not be visible or a metal structure. According to certain aspects of the present teaching, the center portion connector may lie within a substantially vertical plane or a substantially horizontal plane or may be offset at a certain angle with respect to a substantially vertical plane or a substantially horizontal plane.

It is noted that the features described above with respect to the convex portion of FIGS. 16A-16G, 17A-17G, 18A-18G, and 19A-19G may also be applicable to any of the other embodiments disclosed herein.

It is further noted that the periodontal prosthetic in the embodiments disclosed herein may be also be made of a hardened resin material, which provides inherent structural support to the overall device.

Also provided are methods for making various embodiments of the cosmetic facial prosthesis comprising providing or forming a resin material, shaping the resin material and allowing the resin material to harden into the shape of any of the embodiments disclosed herein.

Numerous embodiments have been described herein. It will be apparent to those skilled in the art that the above devices, apparatuses and methods may incorporate changes and modifications without departing from the general scope of this invention. It is intended to include all such modifications and alterations in so far as they come within the scope of the appended claims or the equivalents thereof. The phrase “associated with” as used in this document, refers to structures which support the disclosed device and may also refer to structures not disclosed herein capable of supporting the disclosed device. Further, the “invention” as that term is used in this document is what is claimed in the claims of this document. The right to claim elements and/or sub-combinations that are disclosed herein as other inventions in other patent documents is hereby unconditionally reserved.

Having thus described the disclosed apparatus and method, it is now claimed:

What is claimed is:

1. A cosmetic facial prosthesis comprising:
a periodontal prosthetic having a front side and a back side that fits over a user's upper or lower jaw bone, wherein the front side of the periodontal prosthetic fits over the user's front gingivae and the back side of the periodontal prosthetic fits over the back side of the user's back gingivae;
at least one opening within the periodontal prosthetic which is capable of fitting over one or more of a user's teeth as the periodontal prosthetic lies over a user's gingivae; and
a convex portion on the periodontal prosthetic that extends in a distal direction toward the gingiva and extends outward within the vestibule in a buccal direction.

2. The cosmetic facial prosthesis of claim 1, wherein the periodontal prosthetic comprises a left side, a center side and a right side, wherein the left side comprises a front left side and a back left side, the center side comprises an optional front center side and a back center side and the right side comprises a front right side and a back right side.

3. The cosmetic facial prosthesis of claim 2, further comprising an internal hook which extends within the periodontal prosthetic or an external hook which extends outward from the periodontal prosthetic and which is capable of attaching the cosmetic facial prosthesis to a user's tooth or to the upper or lower jaw bone of the user.

4. The cosmetic facial prosthesis of claim 3, further comprising a wire or wire-like support structure embedded within the periodontal prosthetic that provides structural support to the cosmetic facial prosthesis.

5. The cosmetic facial prosthesis of claim 3, wherein the internal or external hook is in the form of a resin structure, a resin structure mimicking the appearance of the user's gingivae, a wire or wire-like structure embedded within a resin structure so as to not be visible or positioned on the back side of the user's mouth so as to not be visible or is a metal structure which extends outward from the periodontal prosthetic.

6. The cosmetic facial prosthesis of claim 2, wherein the front center side is excluded and the back center side comprises a wire or wire-like structure that connects the left side of the cosmetic facial prosthesis to the right side of the cosmetic facial prosthesis.

7. The cosmetic facial prosthesis of claim 6, further comprising a first opening between the front and back left side of the cosmetic facial prosthesis and a second opening between the front and back right side of the cosmetic facial prosthesis, wherein the first opening extends the entire length of the left side of the cosmetic facial prosthesis and the second opening extends the entire length of the right side of the cosmetic facial prosthesis.

8. The cosmetic facial prosthesis of claim 6, further comprising a plurality of individual tooth openings between the front and back left side of the cosmetic facial prosthesis and a plurality of individual tooth openings between the front and back right side of the cosmetic facial prosthesis that allow the cosmetic facial prosthesis to fit over individual teeth.

9. The cosmetic facial prosthesis of claim 1, wherein the periodontal prosthetic comprises a left side, a right side, a front side and a back side that is capable of fitting over the upper or lower jaw bone within the right side, center side or left side of a buccal cavity.

10. The cosmetic facial prosthesis of claim 9, further comprising a first external hook which extends outward from the back right side of periodontal prosthetic and a second external hook which extends outward from the back left side of the periodontal prosthetic, wherein the first and second external hooks are capable of attaching the cosmetic facial prosthesis to a user's tooth or to the upper or lower jaw bone of the user.

11. The cosmetic facial prosthesis of claim 10, further comprising an opening extending the entire length of the cosmetic facial prosthesis or a plurality of individual tooth openings between the front and back side of the cosmetic facial prosthesis that allow the cosmetic facial prosthesis to fit over individual teeth and wherein the cosmetic facial prosthesis optionally comprises an internal hook which extends within the opening spanning the entire length of the cosmetic facial prosthesis or which extends between the individual tooth openings between the front and back side of the cosmetic facial prosthesis, optionally, wherein the hook is connected to a wire or wire-like extension which extends from the back left or back right side of the cosmetic facial prosthesis, wherein the internal or external hook and wire or wire-like extension is in the form of a resin structure, a resin structure mimicking the appearance of the user's gingivae, a wire or wire-like structure embedded within a resin structure so as to not be visible or positioned on the back side of the user's mouth so as to not be visible or is a metal structure which extends outward from the periodontal prosthetic.

12. The cosmetic facial prosthesis of claim 2,
wherein the front center side or a portion of the front center side, front left side or front right side is excluded and the back center side comprises a connecting surface or a wire or wire-like structure that connects the left side of the cosmetic facial prosthesis to the right side of the cosmetic facial prosthesis,
further comprising an opening which i) extends the entire length of the left side and/or right side of the cosmetic facial prosthesis or ii) a plurality of individual tooth openings between the front and back left side of the cosmetic facial prosthesis and/or a plurality of individual tooth openings between the front and back right side of the cosmetic facial prosthesis that allow the cosmetic facial prosthesis to fit over individual teeth,
further comprising a first external hook which extends outward from the front right side of periodontal prosthetic and a second external hook which extends outward from the front left side of the periodontal prosthetic, wherein the first and second external hooks are capable of attaching the cosmetic facial prosthesis to a user's tooth or to the upper or lower jaw bone of the user, wherein the external hooks are in the form of a resin structure, a resin structure mimicking the appearance of the user's gingivae, a wire or wire-like structure embedded within a resin structure so as to not be visible or is a metal structure which extends outward from the periodontal prosthetic.

13. The cosmetic facial prosthesis of claim 2, further comprising a top surface teeth covering which extends over the left side, the center side and right side of the periodontal prosthetic and connects to the front and back left side, front and back center side and front and back right side of the periodontal prosthetic, further comprising a front surface teeth covering and a back surface teeth covering which extends downward from the top surface teeth covering, wherein the cosmetic facial prosthesis comprises a bottom opening which renders the cosmetic facial prosthesis capable of fitting over the teeth and upper or lower jaw bone, optionally, wherein the cosmetic facial prosthesis further comprises an opening within a portion of the front side capable of exposing one or more teeth.

14. The cosmetic facial prosthesis of claim 13, wherein the front side opening is within the front center side, wherein a center connector portion lies below the front center side opening and connects the left side of the prosthetic facial prosthesis to the right side of the prosthetic facial prosthesis.

15. The cosmetic facial prosthesis of claim 14, further comprising a convex portion positioned below the front surface teeth covering or upward above the front surface teeth covering depending on whether the prothesis is placed on the lower or upper jaw, wherein the convex portion extends in a distal direction toward the gingiva and extends outward within the vestibule in a buccal direction.

16. The cosmetic facial prosthesis of claim 15, wherein the convex portion extends outward from the bottom surface of the teeth covering at at least one angle, wherein the at least one angle is at least about 100° or more.

17. The cosmetic facial prosthesis of claim 16, wherein the front left side, front center side and front right side and convex portion of the periodontal prosthesis comprises a single thickness or a plurality of different thicknesses, wherein the thickness comprises about 3 mm or more.

18. The cosmetic facial prosthesis of claim 2, wherein the front center side is excluded and the back side comprises a concave connecting surface that connects the left side of the cosmetic facial prosthesis to the right side of the cosmetic facial prosthesis, wherein the convex portion extends along the entire length of the left side and the entire length of the right side of the periodontal prosthetic, wherein the convex portion extends outward from the bottom surface of the teeth covering at least one angle, wherein the at least one angle is at least about 100° or more, and wherein the front left side, front center side and front right side and convex portion of the periodontal prosthesis comprises a single thickness or a plurality of different thicknesses, wherein the thickness comprises about 3 mm or more.

19. The cosmetic facial prosthesis of claim 2, wherein the front center side is excluded and the back side comprises a wire or wire-like connector that connects the left side of the cosmetic facial prosthesis to the right side of the cosmetic facial prosthesis, wherein the convex portion extends along the entire length of the left side and the entire length of the right side of the periodontal prosthetic, wherein the periodontal prosthesis comprises at least one internal hook which extends within the space between the front left side and the back left side and/or the space between the front right side and back right side of the periodontal prosthesis, wherein the wire or wire-like connector and/or the internal hooks are in the form of a resin structure, a resin structure mimicking the appearance of the user's gingivae, a wire or wire-like structure embedded within a resin structure so as to not be visible or is a metal structure, and wherein the cosmetic facial prosthesis does not include a top surface teeth covering, a front surface teeth covering and a back surface teeth covering.

20. The cosmetic facial prosthesis of claim 19, wherein the convex portion extends along the entire length of the left side and the entire length of the right side of the periodontal prosthetic, wherein the convex portion extends outward from the bottom surface of the teeth covering at least one angle, wherein the at least one angle is at least about 100° or more, and wherein the front left side, front center side and front right side and convex portion of the periodontal prosthesis comprises a single thickness or a plurality of different thicknesses, wherein the thickness comprises about 3 mm or more.
